(19) United States
(12) Patent Application Publication
(10) Pub. No.: US 2007/0016123 A1
(43) Pub. Date: Jan. 18, 2007

(54) Wound dressing having a folded release sheet

(75) Inventor: Jarl Jensen, Nyack, NY (US)

Correspondence Address: THE ECLIPSE GROUP, 10605 BALBOA BLVD., SUITE 300, GRANADA HILLS, CA, 91344, US

(73) Assignee: Jentec, Inc., Northvale, NJ (US)

(21) Appl. No.: 11/183,435

(22) Filed: Jul. 18, 2005

(51) Int. Cl. A61F 15/00; A61F 13/00

(52) U.S. Cl. 602/57

(57) ABSTRACT

A wound dressing having a dressing layer covered by backing layer on one side. The opposite side is a skin-contacting surface covered by a first release sheet and a second release sheet. The first release sheet is folded into an area that covers the dressing layer in a first release sheet region. The other side of the fold provides a pull tab area that permits removal of the first release sheet from the dressing layer. The second release sheet covers the skin-contacting surface in a second release sheet region.

### A. FIELD OF THE INVENTION

The present invention relates to the field of dressings that are applied to skin, and more particularly, to the field of dressings having components for assisting a user in applying the dressing to the skin.

### B. DESCRIPTION OF RELATED ART

Wound dressings typically comprise a dressing portion, an adhesive, and protective liners. The dressing portion is the part of the wound dressing that makes protective contact with the wound by keeping it clean and by protecting it from exposure to bacteria and other harmful agents in the environment. The adhesive material maintains the dressing portion on the skin. The protective liners protect the dressing portion from bacteria and other contaminants.

Makers of wound dressings have endeavored to make it as easy as possible for a user to apply a wound dressing to a wound site. Users, such as nurses, typically attempt to apply a dressing to a wound as quickly as possible in order to bring a healing-promoting environment to the wound quickly and to stop the bleeding. From an efficiency standpoint, nurses may have many other tasks to perform and would further benefit from dressings that require as few steps as possible to apply to the wound.

With many wound dressings, the ease of applying a wound dressing depends on the manner in which a surface of the dressing portion is exposed so that it may be applied to the skin. This involves removing one of the protective liners that are applied to the dressing layer during manufacture and packaging. Most wound dressings, however, are made to be sterile until it is applied so that a wound is covered with a sterile environment. Often, in applying a wound dressing, the user may touch the surface of the dressing and possibly contaminate the dressing layer.

It would be desirable to have a dressing that may be used on the human skin that is easy to apply without contaminating the dressing during application.

### SUMMARY

In accordance with embodiments consistent with the present invention, a wound dressing is provided in which a dressing layer having a skin-contacting surface and an adhesive composition is characterized by a first release sheet region and a second release sheet region. The dressing layer has a backing layer substantially covering a surface of the dressing layer opposite the skin-contacting surface. A first release sheet is folded to divide it into a pull tab area extending from the fold and a dressing cover area to cover the first release sheet region. The fold defines a boundary between the first release sheet region and the second release sheet region. A second release sheet having an area greater than the second release sheet region covers the skin-contacting surface on the second release sheet region.

The above-mentioned and other features, utilities, and advantages of the invention will become apparent from the following detailed description of exemplary embodiments of the invention together with the accompanying drawings.

Other embodiments, features, and advantages of the invention will become apparent to one with skill in the art upon examination of the following figures and detailed description. It is intended that all such additional embodiments, features, and advantages be included within this description, be within the scope of the invention, and be protected by the accompanying claims.

### BRIEF DESCRIPTION OF THE DRAWINGS

Presently preferred embodiments of the invention are described below in conjunction with the appended drawing figures, wherein like reference numerals refer to like elements in the various figures, and wherein:

FIG. 1 depicts a perspective view of a dressing according to a preferred embodiment of the present invention.

FIG. 2 depicts a cross-sectional view of the dressing of FIG. 1.

FIG. 3 depicts a cross-sectional view of the dressing of FIG. 1 with the components separated from one another.

FIG. 4 depicts an overhead view of the dressing of FIG. 1.

FIGS. 5A-5G depicts an example of how the dressing of FIG. 1 may be applied on to a wound bed.

### DETAILED DESCRIPTION

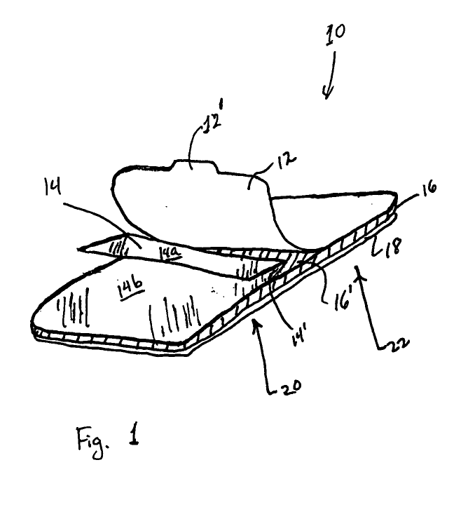

FIG. 1 shows a perspective view of a wound dressing 10 according to an exemplary embodiment of the present invention. The wound dressing 10 comprises a dressing layer 16 covered by a first release sheet 14 and a second release sheet 12 on a skin-contacting surface 16′ of the dressing layer 16. The dressing layer 16 is covered by a backing layer 18 on the side opposite the skin-contacting surface 16′. The dressing layer 16 has a first release sheet region 20 substantially covered by the first release sheet 14 and a second release sheet region 22 substantially covered by the second release sheet 12.

The first release sheet 14 has a fold 14′ that divides the first release sheet 14 into a pull tab area 14a extending from the fold and a dressing cover area covering the first release sheet region 20 of the dressing layer 16. The pull tab area 14a provides a grip that allows a user to pull on the tab to remove the first release sheet 14 from the skin-contacting surface 16′ of the dressing layer 16.

The second release sheet 12 extends over the skin-contacting surface 16′ of the dressing layer 16 on the second release sheet region 22. The area of the second release sheet 12 is generally greater than, and preferably substantially equal to, the area of the second release sheet region 22. The portion of the second release sheet 12 that extends beyond the second release sheet region 22 may be used as a grip to pull on the second release sheet 12 and remove it from the skin-contacting surface 16′. In one exemplary embodiment, a grip tab 12′ extends from an edge of the portion of the second release sheet 12 that extends beyond the second release sheet region 22.

The above-described configuration of the wound dressing 10 advantageously simplifies the process of applying the dressing layer 16 to a wound site. A user of the wound dressing 10 may apply the wound dressing 10 by gripping the grip tab 12′ on the second release sheet 12 and pulling on the pull tab area 14′ of the first release sheet 14. The user may then place the exposed portion of the dressing layer 16 to one side of the wound and peel off the second release sheet 12 while covering the wound with the remainder of the dressing layer 16 as it becomes exposed. Application of the wound dressing 10 may therefore be accomplished without requiring the user to touch the skin-contacting surface 16′ in an easy, efficient manner.

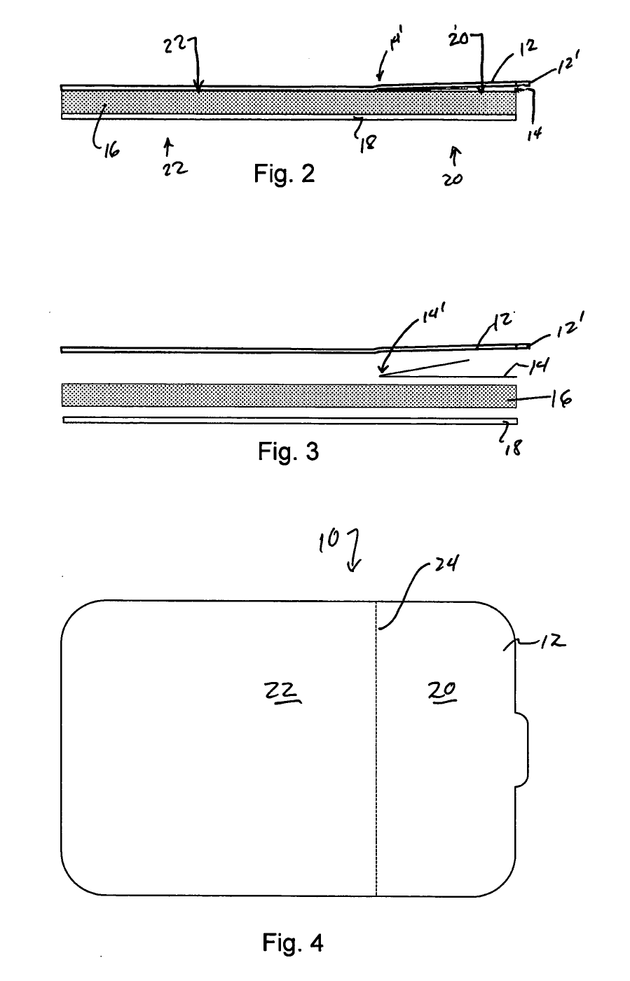

FIG. 2 depicts a cross-sectional view of the wound dressing 10 in FIG. 1 in accordance with an exemplary embodiment. As shown in FIG. 2, the wound dressing 10 is preferably flat to allow for packaging in an envelope (not shown) that may be sealed to protect the wound dressing 10 from outside contaminants. The wound dressing 10 is preferably assembled and packaged in a sterile environment, such as a clean room. The sealed envelope keeps the wound dressing 10 sterile until the user applies the wound dressing 10 to a wound site. The configuration of the wound dressing 10 allows the user to apply the dressing 10 without touching the skin-contacting surface 16′ thereby allowing the dressing 10 to remain sterile as it is applied.

Referring to FIG. 2, the second release sheet 12 substantially covers the area of the dressing layer 16 and makes contact with the skin-contacting surface 16′ in the second release sheet region 22. The first release sheet 14 covers the dressing layer and makes contact with the skin-contacting surface 16′ in the first release sheet region 20. The fold 14′ in the first release sheet 14 serves as a boundary that divides the first release sheet region 20 from the second release sheet region 22.

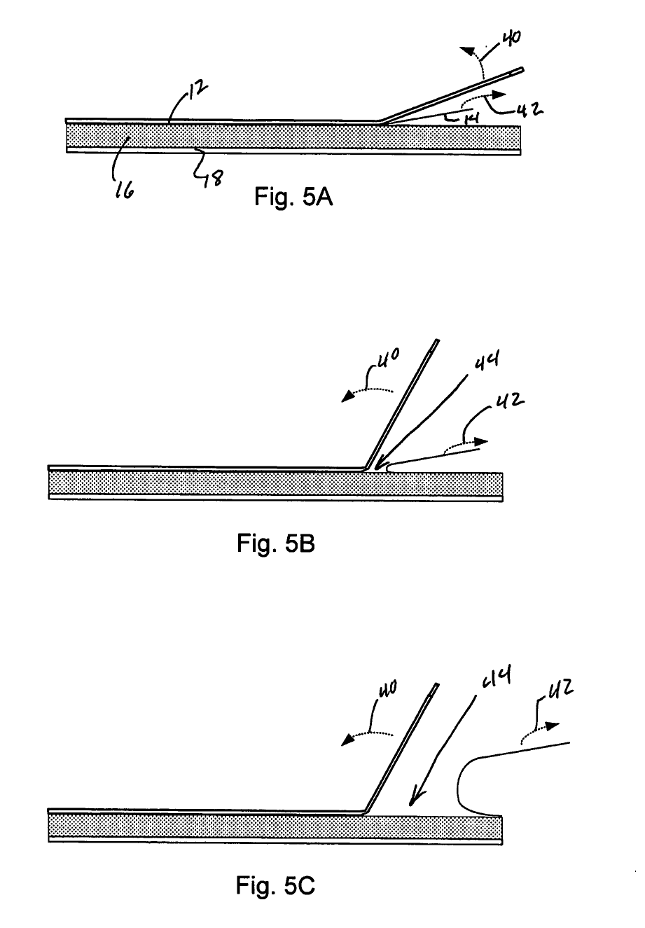

FIG. 3 is a cross-sectional view of the wound dressing 10 with its component layers separated. FIG. 3 illustrates more clearly how the components fit together to form the wound dressing 10.

The first release sheet 14 is preferably made of a film material having an adhesive resistant surface on the dressing cover area, which is the portion that contacts the dressing layer 16. The first release sheet material should provide flexibility and make the sheet sufficiently rigid to protect the dressing layer portion it covers. In exemplary embodiments, the first release sheet 14 is made of one, or a combination, of plastic, mylar, PET, and paper. The adhesive resistant surface may be a layer of a silicone-based material applied to the film material.

The second release sheet 12 is preferably made of a protective material having an adhesive resistant surface on the side covering the second release sheet region 22 of the skin-contacting surface 16′ of the dressing layer 16. The first release sheet material should provide flexibility and make the sheet sufficiently rigid to protect the dressing layer portion it covers. The protective material may be a material selected from the group consisting of paper, cardboard, PET, plastic, and mylar. The adhesive resistant surface may be a layer of a silicone-based material applied to the film material. In a preferred embodiment, the second release sheet 12 is made of siliconized paper.

The backing layer 18 may be permanently adhered to the dressing layer 16 thereby providing a protective layer for the wound dressing 10 as the dressing covers a wound site. The backing layer 18 is preferably made of a transparent material that allows a user to see the wound while the wound dressing 10 covers the wound site. Alternatively, the backing layer 18 may be an adhesive resistant material that may be removed upon application to the wound site. In exemplary embodiments, the backing layer 18 may be made of polyurethane, polyethylene, polypropylene, styrene-isoprene copolymers, styrene-butadiene block copolymers, butadiene rubbers, isoprene rubbers, neoprene rubbers, acrylonitrile rubbers, silicone rubbers, butyl rubbers, chloroprene rubbers, polyvinylchloride, polyamides, or mixtures thereof.

The dressing layer 16 preferably combines an adhesive material with a material having high moisture absorption properties. The adhesive material secures the dressing layer 16 to the skin at the wound site. The adhesive material may include a tackifier, such as a hydrocarbon resin. In one exemplary embodiment, the adhesive material comprises a combination of a tackifier and hot melt acryclic.

The highly moisture-absorbent material may be a hydrocolloid material. One example of a hydrocolloid that may be used is calcium carboxymethylcellulose (“CMC”). Others include pectin, gelatin, high molecular weight carbowax, carboxypolymethylene, polyacrylate, polyvinyl alcohol, and polyvinyl pyrrolidone.

In exemplary embodiments, the tackifier is preferably a hydrocarbon resin. The elastomer is preferably a styrene-olefin-styrene compound, but may also be polyisobutylene, natural rubber, silicone rubber, arcylonitrile rubber, and polyurethane rubber. The dressing layer 16 may also include an extender, preferably paraffin oil. The extender may also be a material that functions as a plasticizer, particularly in combination with the elastomer. Such plasticizers include glycerin (glycerol), sorbitol, triethylene glycol. The extender may also be mineral oil.

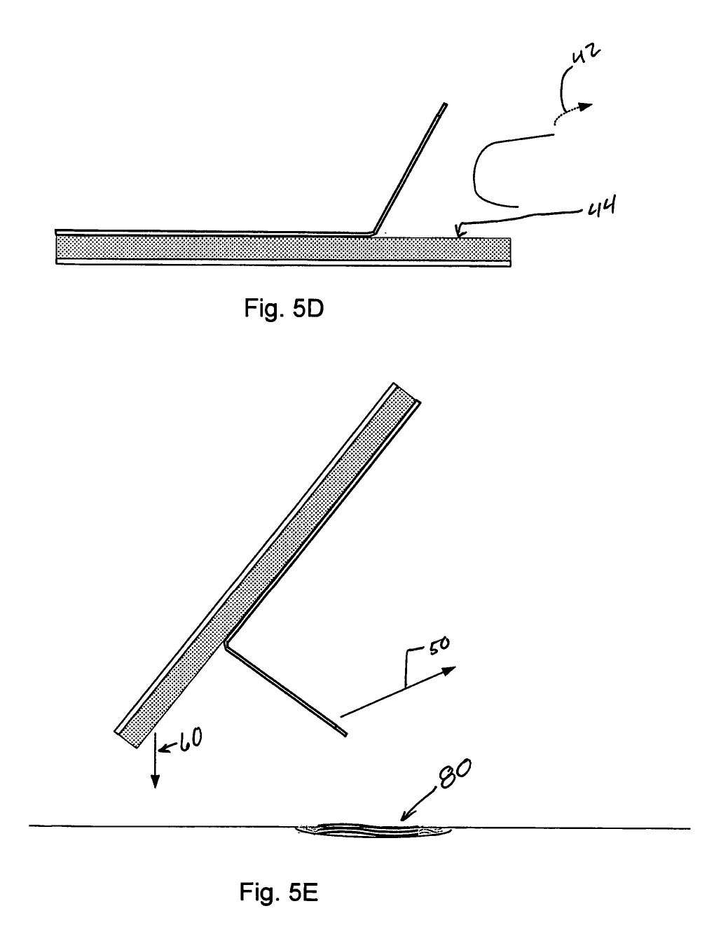

FIG. 4 shows an overhead view of the wound dressing 10 of FIG. 1 from above the second release sheet 12. FIG. 4 shows a dividing line 24 at the location of the fold 14′ in the first release sheet 12 divides the dressing layer 16 into the first release sheet region 20 and the second release sheet region 22.

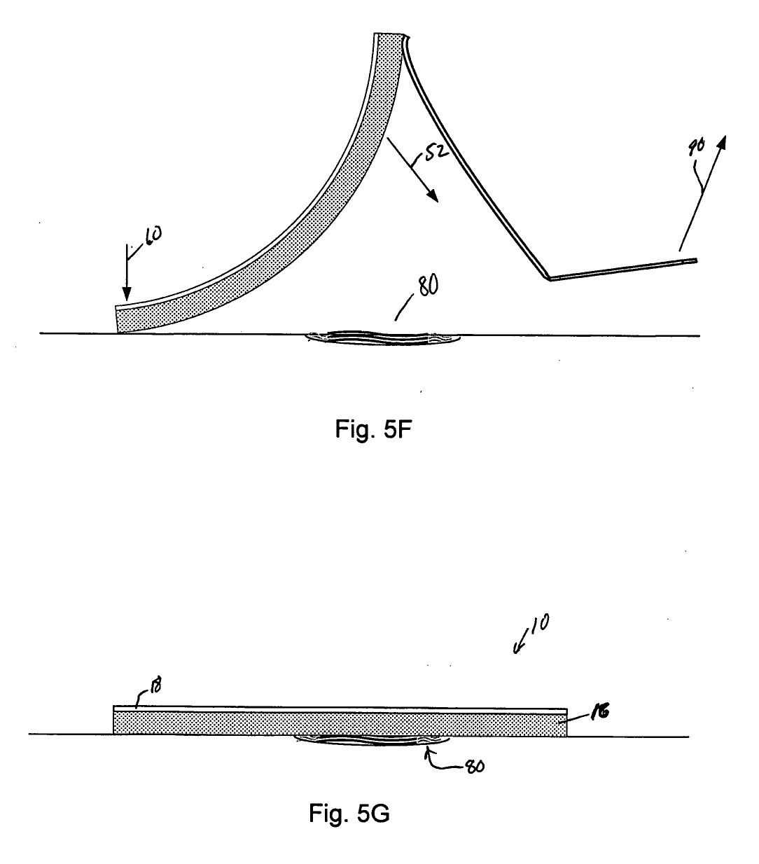

FIGS. 5A-5G illustrate how the wound dressing 10 may be applied to a wound site. As shown in FIG. 5A, a user separates the second release sheet 12 from the area of the first release sheet 14 by pulling on the second release sheet 14 in the direction shown by arrow 40. The user may then grip the first release sheet 14 and pull in the direction shown by arrow 42 to begin removing the first release sheet 14.

FIG. 5B depicts the manner in which an exposed area 44 on the dressing layer 16 begins to emerge from the pulling of the first release sheet 12 in the direction shown by arrow 42 and the pulling of the second release sheet 14 in the direction shown by arrow 40. FIG. 5C shows an even larger exposed area 44 as the first release sheet 42 continues to come off in the direction shown by arrow 42. FIG. 5D shows the completely exposed area 44 of the dressing layer 16 in the first release sheet region 20 after the complete removal of the first release sheet 14.

With the first release sheet 14 completely removed, the user may turn the wound dressing 10 upside down and begin to apply the wound dressing 10 as shown in FIG. 5E. An exposed part of the dressing layer is pressed down on the skin as shown by the arrow 60. The user may then peel off the second release sheet 12 by pulling in the direction shown by the arrow 90 exposing more of the dressing layer. Referring to FIG. 5F, as more of the dressing layer is exposed, the dressing layer is moved in the direction shown by arrow 52 towards a wound site 82. When the second release sheet 12 is completely removed, the dressing layer 16 may then fully rest on the wound site 80 as shown in FIG. 5G. The wound dressing 10 is now resting on the wound site 80 and the dressing layer 16 is covering the wound site 80 providing it with moisture absorbing capabilities. Moreover, because the dressing 10 was applied without the need for the user to touch the skin-contacting surface 16′ of the dressing layer 16, the dressing 10 is still substantially sterile providing a clean environment that promotes healing at the wound site.

Exemplary embodiments of the present invention have been described. Persons of ordinary skill in the art will appreciate that variations may be made without departure from the scope and spirit of the invention. For example, one of ordinary skill in the art will appreciate that the wound dressing 10 may be of any shape or size. This true scope and spirit is defined by the appended claims, interpreted in light of the foregoing.

We claim:

1. A wound dressing comprising
a dressing layer having a skin-contacting surface and an adhesive composition, the skin-contacting surface having a first release sheet region and a second release sheet region;
a backing layer substantially covering a surface of the dressing layer opposite the skin-contacting surface;
a first release sheet having a fold that divides the first release sheet into a pull tab area extending from the fold and a dressing cover area covering the first release sheet region such that the fold defines a boundary between the first release sheet region and the second release sheet region; and
a second release sheet having an area greater than the second release sheet region, the second release sheet covering the skin-contacting surface on the second release sheet region.

2. The dressing of claim 1 wherein the dressing layer contains a water-absorbable material selected from the group consisting of a hydrocolloid, a hydrogel, a foam, polyacrylate, polyvinyl alcohol, and polyvinyl pyrrolidone.

3. The dressing of claim 1 wherein the dressing layer contains a hydrocolloid material selected from the group consisting of: calcium carboxymethylcellulose (“CMC”), pectin, gelatin, high molecular weight carbowax, carboxypolymethylene.

4. The dressing of claim 1 wherein the backing layer is made of a material selected from the group consisting of polyurethane, polyethylene, polypropylene, styrene-isoprene copolymers, styrene-butadiene block copolymers, butadiene rubbers, isoprene rubbers, neoprene rubbers, acrylonitrile rubbers, silicone rubbers, butyl rubbers, chloroprene rubbers, polyvinylchloride, polyamides, or mixtures thereof.

5. The dressing of claim 1 wherein the first release is made of a film material having an adhesive resistant surface on the dressing cover area, the film material selected from the group consisting of a plastic, a mylar, PET and paper.

6. The dressing of claim 5 wherein the adhesive resistant surface comprises a silicone-based layer.

7. The dressing of claim 1 wherein the second release sheet is made of a protective material having an adhesive resistant surface on the side covering the second release sheet region of the skin-contacting surface of the dressing layer, the protective material selected from the group consisting of paper, cardboard, PET, mylar and plastic.

8. The dressing of claim 1 wherein the second release sheet has an area substantially equal to the area of the first release sheet region and the second release sheet region.

9. The dressing of claim 1 wherein the second release sheet comprises a grip tab extending from an edge of the second release sheet opposite the portion of the second release sheet that covers the second release sheet region.

10. The dressing of claim 8 wherein the adhesive resistant surface comprises a silicone-based layer.
